(19) United States
(12) Patent Application Publication
(10) Pub. No.: US 2023/0151084 A1
(43) Pub. Date: May 18, 2023

(54) COMPOSITIONS AND METHODS OF MODULATING B CELL RESPONSE

(71) Applicant: CASE WESTERN RESERVE UNIVERSITY, Cleveland, OH (US)

(72) Inventor: M. Edward Medof, Moreland Hills, OH (US)

(21) Appl. No.: 18/099,048

(22) Filed: Jan. 19, 2023

Related U.S. Application Data
(63) Continuation of application No. 15/179,540, filed on Jun. 10, 2016.
(60) Provisional application No. 62/173,657, filed on Jun. 10, 2015.

(51) Int. Cl. C07K 16/18; C12N 15/113; C07K 16/40

(52) U.S. Cl. CPC C07K 16/18; C12N 15/1138; C07K 16/40; C07K 2317/76; C12N 2310/14

(57) ABSTRACT

A method of treating a B cell mediated disorder in a subject in need thereof, the method includes administering to B cells of the subject at least one agent that inhibits C3 aR and/or C5 aR signaling of the B cells.

### RELATED APPLICATION

This application claims priority from U.S. Provisional Application No. 62/173,657, filed Jun. 10, 2015, the subject matter of which is incorporated herein by reference in its entirety.

### TECHNICAL FIELD

This application relates to a compositions and methods of modulating B cell response and to compositions and methods of treating B cell mediated disorders and conditions.

### BACKGROUND

B cells play a central role in immunity. They have two major functions: 1) They produce antibody. 2) They serve as antigen presenting cells (APCs) that, by virtue of uptake by their surface bound antibody (B cell receptor (BCR)0 process their cognate antigen with much higher efficiency than dendritic cells (DCs) and present it to T cells.

Deficient or deregulated B cell responses are connected with hypo-immune and auto-immune disorders and B cell responses that are elicited by vaccines are needed to prevent numerous infectious and other diseases. Among disorders connected with inadequate B cell responses are Bruton's-hypoglobulinemia and HIV-AIDs, and among those connected with abnormally heightened B cell responses are systemic lupus erythematosus (SLE) and myasthenia gravis (MG). Protective vaccination strategies currently are intensively being sought for Ebola virus and for Malaria falciparum among many other infectious agents. Consequently, the ability to repress or to boost B cell responses has broad clinical relevance.

### SUMMARY

Embodiments described herein relate to compositions and methods of modulating B cell responses to treat B cell mediated disorders in a subject and/or promote a B cell immunological response. The method can include administering to the B cell at least one agent that modulates C3aR and/or C5aR signaling or signal transduction of the B cells.

In some embodiments, the agent can inhibit C3aR and/or C5aR signaling of the B cells and be administered to the cells at an amount effective to inhibit at least one of activity, growth, proliferation, and antibody production of the B cells. The agent that inhibits C3aR and/or C5aR signaling of the B cells can include a complement antagonist that substantially reduces C3aR and/or C5aR signaling of the B cells, such as by inhibiting the interaction of at least one of C3a or C5a with the C3a receptor (C3aR) and C5a receptor (C5aR).

In some embodiments, the complement antagonist is selected from the group consisting of a small molecule, a polypeptide, and a polynucleotide. In some aspects, the polypeptide includes an antibody directed against at least one of C3, C5, C3 convertase, C5 convertase, C3a, C5a, C3aR, or C5aR. In other aspects, the polypeptide can include decay accelerating factor (DAF) (CD55) that accelerates the decay of C5 convertase and C3 convertase. In some aspects, the polynucleotide includes a small interfering RNA directed against a polynucleotide encoding at least one of C3, C5, C3aR, or C5aR.

Another aspect relates to a method of treating a B cell mediated disorder in a subject. The method includes administering at least one complement antagonist to B cell at an amount effective to substantially inhibit C3a receptor (C3aR) and/or C5a receptor (C5aR) signal transduction of the B cells.

In some embodiments, the B cell mediated disorder is an autoimmune disorder selected from the group consisting of systemic lupus erythematosus, Sjogren's syndrome, scleroderma, rheumatoid arthritis, juvenile idiopathic arthritis, graft versus host disease, dermatomyositis, type I diabetes mellitus, Hashimoto's thyroiditis, Graves's disease, Addison's disease, celiac disease, Crohn's Disease, pernicious anaemia, Pemphigus vulgaris, Vitiligo, autoimmune haemolytic anaemia, idiopathic thrombocytopenic purpura, giant cell arteritis, Myasthenia gravis, multiple sclerosis (MS), preferably relapsing-remitting MS (RRMS), glomerulonephritis, Goodpasture's syndrome, bullous pemphigoid, colitis ulcerosa, Guillain-Barre syndrome, chronic inflammatory demyelinating polyneuropathy, Anti-phospholipid syndrome, narcolepsy, sarcoidosis, and Wegener's granulomatosis.

In other embodiments, the B cell mediated disorder is a B cell derived leukemia and/or lymphoma.

Other embodiments relate to a method of stimulating a B cell response in a subject in need thereof by administering to B cells of the subject at least one complement agonist at an amount effective to substantially promote C3a receptor (C3aR) and/or C5a receptor (C5aR) signal transduction of the B cells.

In some embodiments, the stimulation of a B cell response can be used to promote antibody production and/or immunogenic response to an antigen or vaccine administered to the subject.

### BRIEF DESCRIPTION OF THE DRAWINGS

The foregoing and other features of the invention will become more apparent upon a consideration of the following description taken in connection with the accompanying drawings wherein:

FIGS. 1(A-C) illustrate graphs and a schematic drawing showing antibody production is disrupted in C3ar1^{-/-}C5ar1^{-/-} mice and potentiated in Daf1^{-/-} mice.

FIGS. 2(A-B) illustrate graphs showing CSR is disabled in C3ar1^{-/-}C5ar1^{-/-} mice and potentiated in Daf1^{-/-} mice.

FIGS. 3(A-B) illustrate graphs showing BAFF and APRIL viability signaling in B cells depends on C3aR/C5aR signaling.

FIGS. 4(A-B) illustrate a graph and schematic drawing showing B cell proliferation depends on autocrine C3aR/C5aR signaling in B cells.

FIG. 5 illustrates a schematic drawing showing a murine model of SLE.

FIGS. 6(A-D) illustrate graphs and a schematic drawing showing anti-nuclear antibody, IFN-α, and IL-6 expression is abolished if C3aR/C5aR signaling is disabled.

FIGS. 7(A-B) illustrate a graph and schematic drawing showing lymphoid nodules and splenic hyperplasia are repressed is C3aR/C5aR signaling is disabled.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Edition, Springer-Verlag: N.Y., 1991, and Lewin, Genes V, Oxford University Press: New York, 1994. The definitions provided herein are to facilitate understanding of certain terms used frequently herein and are not meant to limit the scope of the application described herein.

The term “polypeptide” refers to an oligopeptide, peptide, or protein sequence, or to a fragment, portion, or subunit of any of these, and to naturally occurring or synthetic molecules. The term “polypeptide” also includes amino acids joined to each other by peptide bonds or modified peptide bonds, i.e., peptide isosteres, and may contain any type of modified amino acids. The term “polypeptide” also includes peptides and polypeptide fragments, motifs and the like, glycosylated polypeptides, and all “mimetic” and “peptidomimetic” polypeptide forms.

The term “polynucleotide” refers to oligonucleotides, nucleotides, or to a fragment of any of these, to DNA or RNA (e.g., mRNA, rRNA, tRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, siRNAs, microRNAs, and ribonucleoproteins. The term also encompasses nucleic acids, i.e., oligonucleotides, containing known analogues of natural nucleotides, as well as nucleic acid-like structures with synthetic backbones.

The term “antibody” refers to whole antibodies, e.g., of any isotype (IgG, IgA, IgM, IgE, etc.), and includes fragments thereof which are also specifically reactive with a target polypeptide. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain polypeptide. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term “antibody” also includes polyclonal, monoclonal, or other purified preparations of antibodies, recombinant antibodies, monovalent antibodies, and multivalent antibodies. Antibodies may be humanized, and may further include engineered complexes that comprise antibody-derived binding sites, such as diabodies and triabodies.

The term “B cell” (also known as a “B lymphocyte”) refers to immune cells which express a cell surface immunoglobulin molecule and which, upon activation, terminally differentiate into cells, which secrete antibody. Accordingly, this includes, for example, convention B cells, CD5 B cells (also known as B-1 cells and transitional CD5 B cells). “B cell” should also be understood to encompass reference to B cell mutants. “Mutants” include, but are not limited to, B cells which have been naturally or non-naturally modified, such as cells which are genetically modified. Reference to “B cells” should also be understood to extend to B cells which exhibit commitment to the B cell image. These cells may be at any differentiative stage of development and therefore may not necessarily express a surface immunoglobulin molecule. B cell commitment may be characterized by the onset of immunoglobulin gene re-arrangement or it may correspond to an earlier stage of commitment which is characterized by some other phenotypic or functional characteristic such as the cell surface expression of CD45R, MHCII, CD10, CD19 and CD38. Examples of B cells at various stages of differentiation include early B cell progenitors, early pro-B cells, late pro-B cells, pre-B cells, immature B cells, mature B cells and plasma cells.

The term “B cell response” refers to any one or more of the functional activities, which a B cell, at any differentiative stage of development, is capable of performing. This includes, for example, proliferation, differentiation, immunoglobulin gene rearrangement, immunoglobulin synthesis and secretion and antigen presentation.

The term “B cell mediated disorder” or “B cell disorder” refers to those diseases and conditions that arise from inappropriate replication or activity of B cells. In some embodiments, the B cell mediated disorder is a B cell lymphoma that results from inappropriate replication of B cells. B cell lymphomas are difficult to treat effectively with the currently available medical methods. Other types of B cell mediated disorders which involve inappropriate replication of B cells include chronic and acute B cell leukemias, multiple myelomas, and some non-Hodgkin's lymphomas. Other embodiments include a growing number of human diseases that have been classified as autoimmune disease, where the host's own immune system attacks the host's own tissue, such as multiple sclerosis (MS), systemic lupus erythematosus (SLE), anti-Hu associated paraneoplastic neurological syndromes, autoimmune hepatitis (AIH). Other candidate autoimmune diseases for treatment include rheumatoid arthritis (RA), myasthenia gravis (MG), autoimmune thyroiditis (Hashimoto's thyroiditis), Graves' disease, inflammatory bowel disease, autoimmune uveoretinitis, polymyositis, scleroderma, and certain types of diabetes. The present treatments for these autoimmune diseases do not cure the disease, but instead only ameliorate the symptoms.

The term “complementary” refers to the capacity for precise pairing between two nucleobases of a polynucleotide and its corresponding target molecule. For example, if a nucleobase at a particular position of a polynucleotide is capable of hydrogen bonding with a nucleobase at a particular position of a target polynucleotide (the target nucleic acid being a DNA or RNA molecule, for example), then the position of hydrogen bonding between the polynucleotide and the target polynucleotide is considered to be complementary. A polynucleotide and a target polynucleotide are complementary to each other when a sufficient number of complementary positions in each molecule are occupied by nucleobases, which can hydrogen bond with each other. Thus, “specifically hybridizable” and “complementary” are terms which can be used to indicate a sufficient degree of precise pairing or complementarity over a sufficient number of nucleobases such that stable and specific binding occurs between a polynucleotide and a target polynucleotide.

The term “subject” refers to any warm-blooded organism including, but not limited to, human beings, rats, mice, dogs, goats, sheep, horses, monkeys, apes, rabbits, cattle, etc.

The terms “complement polypeptide” or “complement component” refer to a polypeptide (or a polynucleotide encoding the polypeptide) of the complement system that functions in the host defense against infections and in the inflammatory process. Complement polypeptides constitute target substrates for the complement antagonists provided herein.

The term “complement antagonist” refers to a polypeptide, polynucleotide, or small molecule capable of substantially reducing or inhibiting the activity of a complement component.

A complement component can include any one or combination of interacting blood polypeptides or glycoproteins. There are at least 30 soluble plasma polypeptides, in addition to cell surface receptors, which can bind complement reaction products and which can occur on inflammatory cells and cells of the immune system. In addition, there are regulatory membrane proteins that can protect host cells from accidental complement attack. Complement components can include polypeptides that function in the classical pathway, such as C2, polypeptides that function in the alternative pathway, such as Factor B, and polypeptides that function in the lectin pathway, such as MASP-1.

Complement components can also include: any of the “cleavage products” (also referred to as “fragments”) that are formed upon activation of the complement cascade; complement polypeptides that are inactive or altered forms of complement polypeptides, such as iC3 and C3a-desArg; and components indirectly associated with the complement cascade. Examples of such complement components can include, but are not limited to, C1q, C1r, C1s, C2, C3, C3a, C3b, C3c, C3dg, C3g, C3d, C3f, iC3, C3a-desArg, C4, C4a, C4b, iC4, C4a-desArg, C5, C5a, C5a-des-Arg, C6, C7, C8, C9, MASP-1, MASP-2, MBL, Factor B, Factor D, Factor H, Factor I, CR1, CR2, CR3, CR4, properdin, C1Inh, C4bp, MCP, DAF, CD59 (MIRL), clusterin, HRF, and allelic and species variants of any complement polypeptide.

The terms “treatment,” “treating,” or “treat” refers to any specific method or procedure used for the cure of, inhibition of, prophylaxis of, reduction of, elimination of, or the amelioration of a disease or pathological condition.

As used herein, the term “effective amount” refers to a dosage of an agent described herein administered alone or in conjunction with any additional therapeutic agents that are effective and/or sufficient to provide treatment of a disease or pathological condition. The effective amount can vary depending on the subject, the disease being treated, and the treatment being affected.

The term “therapeutically effective amount” refers to that amount of an agent described herein administered alone and/or in combination with additional therapeutic agents that results in amelioration of symptoms associated with a disease or pathological condition.

The terms “parenteral administration” and “administered parenterally” refers to modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The terms “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

The terms “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, and general safety and purity standards as required by FDA Office of Biologics standards. Supplementary active ingredients can also be incorporated into the compositions.

As used herein, “Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

Embodiments described herein relate to compositions and methods of modulating B cell responses to treat B cell mediated disorders in a subject and/or promote B cell immunological response. The methods can include administering to the B cell at least one agent that modulates (e.g., inhibits or promotes) C3a receptor (C3aR) and/or C5a receptor (C5aR) signaling or signal transduction of the B cells.

It was found that complement anaphylatoxins C3a and C5a, generally believed to derive from serum complement proteins and function principally in innate immunity, are in fact endogenously synthesized by B cells themselves and feedback through C3a and C5a receptors (C3aR/C5aR) on their surfaces to transduce signals needed for B cell activation. This autocrine C3aR/C5aR signaling is required for the survival and growth signaling by the B cell growth factors BAFF and APRIL. It is also required for the proliferation of B cells in response to antigen and CD4⁺T cell help as well as for B cell production of inflammatory cytokines including IL-6. Moreover, it is required for class switch recombination (CSR) which gives rise antibodies of different isotypes, i.e., IgG1,2,3,4, IgE, and IgA, that participate in multiple immune processes.

It was further found that autocrine C3aR/C5aR signaling in B cells is integral to both primary and secondary Ab responses and that inhibition of this signaling ameliorates B cell mediated pathologies, such as systemic lupus erythematosus (SLE). Conversely, it was found that potentiated C3aR/C5aR signaling in B cells augments the antibody response, a finding centrally relevant to improved vaccine development.

Accordingly, based at least in part on these findings, in some embodiments, B cells expressing C3a receptor (C3aR) and C5a receptor (C5aR) can be contacted (e.g., directly or locally) with a therapeutically effective amount of an agent that modulates (e.g., inhibits or promotes) C3aR and/or C5aR signaling of the B cells and modulates (e.g., inhibits or promotes) response of the B cells. This modulation of growth factor response can affect viability, function, proliferation, and antibody production of the B cells and treat diseases, disorders, and conditions where inhibition or promotion of a B cell response is desired.

It should be understood that the B cell, which is the subject of modulation in accordance with the methods described herein, may be an isolated B cell or a B cell which forms part of a group of cells, such as an isolated tissue. The B cell may also be localized in a mammal that is it is not isolated, therefore requiring the subject method to be performed in vivo. Where the subject cell is one of a group of cells or a tissue, either isolated or not, the subject method may modulate the functioning or response of all the B cells in that group or just a subgroup of B cells in that group. Similarly, in the context of the modulation of the biological functioning or development of a mammal, it should be understood that the subject modulation may be achieved in the context of modulating B cell functioning either systemically or in a localized manner. Still further, irrespective of which means is employed, the cellular impact of the change in B cell functioning may occur in the context of either all cells or just a subgroup of cells within the relevant environment.

It should be understood that the B cell which is treated according to the method described herein may be located ex vivo or in vivo. By “ex vivo” is meant that the cell has been removed from the body of a subject wherein the modulation of its activity will be initiated in vitro. For example, the cell may be a B cell which is to be used as a model for studying any one or more aspects of the pathogenesis of autoimmune conditions which are characterized by aberrant B cell activity.

In some embodiments, a B cell response (e.g., growth, proliferation, and/or antibody production) of a B cell can be inhibited by administering to the B cell an agent that inhibits C3aR and/or C5aR signaling of the B cells. The agent can be selected from the group consisting of a complement antagonist that inhibits or substantially reduces the interaction of at least one of C3a or C5a with the C3a receptor (C3aR) and C5a receptor (C5aR).

In some embodiments, the complement antagonist can inhibit or substantially reduce the activity of a complement component. By inhibiting or substantially reducing the activity of a complement component, it is meant that the activity of the complement component may be entirely or partly diminished. For example, an inhibition or reduction in the functioning of a C3/C5 convertase may prevent cleavage of C5 and C3 into C5a and C3a, respectively. An inhibition or reduction in the functioning of C5, C3, C5a and/or C3a polypeptides may reduce or eliminate the ability of C5a and C3a to bind C5aR and C3aR, respectively. An inhibition or reduction in Factor B, Factor D, properdin, Bb, Ba and/or any other protein of the complement pathway that is used in the formation of C3 convertase, C5 convertase, C5, C3, C5a and/or C3a may reduce or eliminate the ability of C5a and C3a to be formed and bind to C5aR and C3aR, respectively. Additionally, an inhibition or reduction in the functioning of a C5aR or C3aR may similarly reduce or eliminate the ability of C5a and C3a to bind C5aR and C3aR, respectively.

In some embodiments, the at least one complement antagonist can include an antibody or antibody fragment directed against a complement component that can affect or inhibit the formation of C3a and/or C5a (e.g., anti-Factor B, anti-Factor D, anti-C5, anti-C3, ant-C5 convertase, and anti-C3 convertase) and/or reduce C5a/C3a-C5aR/C3aR interactions (e.g., anti-C5a, anti-C3a, anti-C5aR, and C3aR antibodies). In one example, the antibody or antibody fragment can be directed against or specifically bind to an epitope, an antigenic epitope, or an immunogenic epitope of a C5, C3, C3a, C5a, C5aR, C3aR, C5 convertase, and/or C3 convertase. The term “epitope” as used herein can refer to portions of C5, C3, C3a, C5a, C5aR, C3aR, C5 convertase, and/or C3 convertase having antigenic or immunogenic activity. An “immunogenic epitope” as used herein can include a portion of a C5, C3, C3a, C5a, C5aR, C3aR, C5 convertase, and/or C3 convertase that elicits an immune response in a subject, as determined by any method known in the art. The term “antigenic epitope” as used herein can include a portion of a polypeptide to which an antibody can immunospecifically bind as determined by any method well known in the art.

Examples of antibodies directed against C5, C3, C3a, C5a, C5aR, C3aR, C5 convertase, and/or C3 convertase are known in the art. For example, mouse monoclonal antibodies directed against C3aR can include those available from Santa Cruz Biotechnology, Inc. (Santa Cruz, Calif.). Monoclonal anti-human C5aR antibodies can include those available from Research Diagnostics, Inc. (Flanders, N.J.). Monoclonal anti-human/anti-mouse C3a antibodies can include those available from Fitzgerald Industries International, Inc. (Concord, Me.). Monoclonal anti-human/anti-mouse C5a antibodies can include those available from R&D Systems, Inc. (Minneapolis, Minn.).

In some embodiments, the complement antagonist can include purified polypeptide that is a dominant negative or competitive inhibitor of C5, C3, C3a, C5a, C5aR, C3aR, C5 convertase, and/or C3 convertase. As used herein, “dominant negative” or “competitive inhibitor” refers to variant forms of a protein that inhibit the activity of the endogenous, wild type form of the protein (i.e., C5, C3, C3a, C5a, C5aR, C3aR, C5 convertase, and/or C3 convertase). As a result, the dominant negative or competitive inhibitor of a protein promotes the “off” state of protein activity. In the context of the present invention, a dominant negative or competitive inhibitor of C5, C3, C3a, C5a, C5aR, C3aR, C5 convertase, and/or C3 convertase is a C5, C3, C3a, C5a, C5aR, C3aR, C5 convertase, and/or C3 convertase polypeptide, which has been modified (e.g., by mutation of one or more amino acid residues, by posttranscriptional modification, by posttranslational modification) such that the C5, C3, C3a, C5a, C5aR, C3aR, C5 convertase, and/or C3 convertase inhibits the activity of the endogenous C5, C3, C3a, C5a, C5aR, C3aR, C5 convertase, and/or C3 convertase.

In some embodiments, the competitive inhibitor of C5, C3, C3a, C5a, C5aR, C3aR, C5 convertase, and/or C3 convertase can be a purified polypeptide that has an amino acid sequence, which is substantially similar (i.e., at least about 75%, about 80%, about 85%, about 90%, about 95% similar) to the wild type C5, C3, C3a, C5a, C5aR, C3aR, C5 convertase, and/or C3 convertase but with a loss of function. The purified polypeptide, which is a competitive inhibitor of C5, C3, C3a, C5a, C5aR, C3aR, C5 convertase, and/or C3 convertase, can be administered to a cell expressing C5aR and/or C3aR.

It will be appreciated that antibodies directed to other complement components used in the formation of C5, C3, C5a, C3a, C5 convertase, and/or C3 convertase can be used in accordance with the method described herein to reduce and/or inhibit interactions C5a and/or C3a with C5aR and C3aR. The antibodies can include, for example, known Factor B, properdin, and Factor D antibodies that reduce, block, or inhibit the formation of C5a and/or C3a.

In some embodiments, the complement antagonist can include RNA interference (RNAi) polynucleotides to induce knockdown of an mRNA encoding a complement component. For example, an RNAi polynucleotide can comprise a siRNA capable of inducing knockdown of an mRNA encoding a C3, C5, C5aR, or C3aR polypeptide.

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response.

As used herein, the term “RNAi construct” is a generic term used throughout the specification to include small interfering RNAs (siRNAs), hairpin RNAs, and other RNA species, which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (I) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences.

The choice of promoter and other regulatory elements generally varies according to the intended host cell. In general, expression vectors of utility in recombinant DNA techniques are often in the form of “plasmids” which refer to circular double stranded DNA loops, which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodie- sters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Examples of a siRNA molecule directed to an mRNA encoding a C3a, C5a, C5aR, or C3aR polypeptide are known in the art. For instance, human C3a, C3aR, and C5a siRNA is available from Santa Cruz Biotechnology, Inc. (Santa Cruz, Calif.). Additionally, C5aR siRNA is available from Qiagen, Inc. (Valencia, Calif.). siRNAs directed to other complement components, including C3 and C5, are known in the art.

In other embodiments, the RNAi construct can be in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects, which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Such hairpin RNAs are engineered in cells or in an animal to ensure continuous and stable suppression of a desired gene. It is known in the art that siRNAs can be produced by processing a hairpin RNA in the cell.

In yet other embodiments, a plasmid can be used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the a recombinant vector can have the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

Examples RNAi constructs that specifically recognize a particular gene or a particular family of genes, can be selected using methodology outlined in detail above with respect to the selection of antisense oligonucleotide. Similarly, methods of delivery RNAi constructs include the methods for delivery antisense oligonucleotides outlined in detail above.

In some embodiments, a lentiviral vector can be used for the long-term expression of a siRNA, such as a short-hairpin RNA (shRNA), to knockdown expression of C5, C3, C5aR, and/or C3aR in cells expressing C3a receptor (C3aR) and C5a receptor (C5aR) and at least one growth factor receptor (e.g., RTK), such as smooth muscle cells, endothelial cells, leukocytes, cancer cells, neural cells, or fibroblasts. Although there have been some safety concerns about the use of lentiviral vectors for gene therapy, self-inactivating lentiviral vectors are considered good candidates for gene therapy as they readily transfect mammalian cells.

It will be appreciated that RNAi constructs directed to other complement components used in the formation of C5, C3, C5a, C3a, C5 convertase, and/or C3 convertase components can be used in accordance with the method described herein to reduce and/or inhibit interactions C5a and/or C3a with C5aR and C3aR on the cells expressing C3a receptor (C3aR) and C5a receptor (C5aR) and at least one growth factor receptor (e.g., RTK), such as smooth muscle cells, endothelial cells, leukocytes, cancer cells, neural cells, or fibroblasts. The RNAi constructs can include, for example, known Factor B, properdin, and Factor D siRNA that reduce expression of Factor B, properdin, and Factor D.

Moreover, it will be appreciated that other antibodies, small molecules, and/or peptides that reduce or inhibit the formation of C5, C3, C5a, C3a, C5 convertase, and/or C3 convertase and/or that reduce or inhibit interactions C5a and/or C3a with C5aR and C3aR on the cells expressing C3a receptor (C3aR) and C5a receptor (C5aR) can be used as a complement antagonist in accordance with the method described herein. These other complement antagonists can be administered to the B cells at amount effective to inhibit a B cell response. Examples of such other complement antagonists include C5aR antagonists, such as AcPhe[Orn-Pro-D-cyclohexylalanine-Trp-Arg, prednisolone, and infliximab (Woodruff et al,. The Journal of Immunology, 2003, 171: 5514-5520), hexapeptide MeFKPdChaWr (March et al., Mol Pharmacol 65:868-879, 2004), PMX53 and PMX205, and N-[(4-dimethylaminophenyl)methyl]-N-(4-isopropylphenyl)-7-methoxy-1,2,3,4-tetrahydronaphthalen-1-carboxamide hydrochloride (W-54011) (Sumichika et al., J. Biol. Chem., Vol. 277, Issue 51, 49403-49407, Dec. 20, 2002), and a C3aR antagonist, such as SB 290157 (Ratajczak et al., Blood, 15 Mar. 2004, Vol. 103, No. 6, pp. 2071-2078).

In some embodiments, the agent that inhibits at least one of C3aR and/or C5aR signaling in B cell may be used to treat disease conditions characterized by aberrant or unwanted B cell response or functioning, such as aberrant or unwanted B cell activation and/or proliferation. Without limiting embodiments described herein to any one theory or mode of action, conditions which may be treated in accordance with the methods described herein include, but are not limited, autoimmune conditions, acute and chronic organ rejection and B cell neoplasias.

In the context of autoimmune disease, conditions which may be treated in accordance with the method of the described herein include but are not limited to:

### Rheumatoid Arthritis

It has been shown that B cells are important in synovial inflammation and have potential as therapeutic targets (Takemura et al., J. Immunol. 2001; 167(8): 4710-4718; Silverman et al, Arthritis Res. Ther. 2003; 5(Suppl 4): S1-S6; Looney et al., Curr. Opin. Rheumatol. 2004; 16: 180-185; Oligino et al., Arthritis Res. Ther. 2003; 5(Suppl 4): S7-S11; Silverman et al., Arthritis Rheum. 2003; 48(6): 1484-1492; Gorman et al., Arthritis Res. Ther. 2003; 5 (Suppl 4): S17-S21). Bone marrow participates in rheumatoid arthritis by generating B cell-rich lesion, which induce endosteal bone formation (Hayer et al., Bone Miner. Res. 2004, 19(6):990-998). The contribution of B cells in rheumatoid arthritis has been validated by data from clinical trials indicating that B cell depletion with rituximab is highly therapeutic (Edwards et al., N. Engl. J. Med. 2004; 350 (25): 2572-2581).

### Multiple Sclerosis

In the past research has largely focused on the contribution of T cells in multiple sclerosis but recent studies are revealing the potential role of B cells in the disease process (Archelos et al., Ann. Neurol. 2000; 47(6): 694-706; Iglesias et al., Glia 2001; 36(2): 220-234; Hemmer et al., Nat. Rev. Neurosci. 2002; 3(4): 291-301; Hemmer et al., Curr. Opin. Neurol. 2002; 15(3): 227-231; Qin et al., Int. MS J. 2003; 10(4): 110-120; Burgoon et al., Front. Biosci. 2004; 1(9): 786-796; Alter et al., J. Immunol. 2003; 170: 4497-4505). The B cell mediated immune response is an early event of the inflammatory reaction in the central nervous system in multiple sclerosis (Qin et al., Lab. Invest. 2003; 83(7): 1081-1088; Haubold et al., Ann. Neurol. 2004, 56(1):97-107). The contribution of B cells may be mainly through demyelination (Svensson et al., Eur. J. Immunol. 2002; 32(7): 1939-1946). Tranilast may inhibit both the inflammatory response and demyelination in multiple sclerosis.

### Systemic Lupus Erythematosus

B cells play a central role in the pathogenesis of systemic lupus erythematosus (SLE) (Looney et al. 2004, supra; Chan et al., Immunol. Rev. 1999; 169: 107-121; Looney et al., Arthritis. Rheum. 2004; 50(8): 2580-2589; Anolik et al., Curr. Opin. Rheumatol. 2004; 16(5): 505-512; Looney et al., Lupus 2004; 13(5): 381-390; Baker et al., Autoimmun. Rev. 2004; 3(5): 368-375; Higuchi et al., J. Immunol. 2002; 168(1): 9-12; Desai-Mehta et al., J. Clin. Invest. 1996; 97(9): 2063-2073). An antibody-independent role of B cells has been demonstrated in murine lupus (Chan et al., J. Immunol. 1999; 163(7): 3592-3596; Chan et al., J. Exp. Med. 1999; 189(10): 1639-1648). This has been confirmed by a significant improvement in disease activity in patients treated with rituximab even in the absence of substantial serologic responses (Looney et al., 2004, supra). The successful treatment of SLE with rituximab demonstrates the value in targeting B cells in this disease (Looney et al., 2004, supra).

### Psoriatic Arthritis

There is evidence that antigen-activated B cells participate in the development of chronic synovitis in psoriatic arthritis (Gerhard et al., Z. Rheumatol. 2002, 61(6):718-727).

### Inflammatory Bowel Disease

The two major forms of inflammatory bowel disease (IBD) are Crohn's disease and ulcerative colitis. The presence of circulating antibodies to colonic epithelial cells has been reported in Crohn's disease and ulcerative colitis (Hibi et al., Clin. Exp. Immunol. 1983; 54(1): 163-168; Takahashi et al., J. Clin. Invest. 1985; 76(1): 311-318; Sadlack et al., Cell 1993; 75(2): 253-261). There is evidence that the pathogenesis of IBD may be triggered by a primarily B cell mechanism through the ectopic expression of the CD40 ligand (CD40L) on B cells (Kawamura et al., J. Immunol. 2004; 172(10): 6388-6397). A similar ectopic expression of CD40L in B cells can induce a lupus-like disease and there is an increased expression of CD4OL by B cells in SLE (Desai-Mehta et al., 1996, supra).

Several reports have described a relationship between IBD and SLE supporting the idea that both IBD and SLE may be triggered by primarily dysregulated B cells (Ishikawa et al., J Dermatol. 1995; 22(4): 289-291; Kritikos et al., Eur. J. Gastroenterol. Hepatol. 1998; 10(5): 437-439). It has also been shown that B cells may play an important role in the development of inflammation in a murine model of Crohn's disease by inhibiting regulatory T cells (Olson et al., J. Clin. Invest. 2004; 114(3): 389-398).

### Type 1 Diabetes

B cells play a critical role as antigen-presenting cells in the development of T cell-mediated autoimmune type 1 diabetes in the nonobese diabetic mouse (Noorchashm et al., Diabetes 1997; 46(6): 941-946; Noorchashm et al., J. Immunol. 1999; 163(2): 743-750; Greeley et al., J. Immunol. 2001; 167(8): 4351-4357; Akashi et al., Int. Immunol. 1997; 9(8): 1159-1164; Serreze et al., J. Exp. Med. 1996; 184(5): 2049-2053; Serreze et al., J. Immunol. 1998; 161(8): 3912-3918; Chiu et al., Diabetes 2001; 50(4): 763-770; Silveira et al, Eur. J. Immunol. 2002; 32(12): 3657-3666; Serreze et al., Curr. Dir. Autoimmun. 2003; 6: 212-227; Silveira et al., J. Immunol. 2004; 172(8): 5086-5094). This collaboration between T cells and B cells in autoimmune diabetes indicates that a drug such as tranilast, which can downregulate B cell functioning will be efficacious. There is also evidence that interventions directed at B cells may be useful in the later stages of the disease (Kendall et al., Euro J. Immunol. 2004; 34(9): 2387-2). This may be important in the treatment of human type 1 diabetes in which early diagnosis and appropriate preventative measures are difficult.

### Psoriasis

Psoriasis is now considered to be a T cell-mediated disease (Morel et al., J Autoimmun. 1992; 5(4): 465-477; Bachelez et al., J Autoimmun. 1998; 11(1): 53-62; Boyman et al., J. Exp. Med. 2004; 199(5): 731-736). Increased B cell infiltration has been reported in the lesional tissue of patients with non-arthritic psoriasis (Griffiths C. E. J. Eur. Acad. Dermatol. Venerol. 2003; 17(Suppl 2): 1-5). There are associations between psoriasis and Crohn's disease (Sarwal et al., N. Engl. J. Med. 2003; 349(2): 125-138). B cells are involved in the pathology of Crohn's disease and may contribute to the development of psoriasis (Olson et al., 2004, supra).

### Graves' Disease, Hashimoto's Thyroiditis and Autoimmune Thyroiditis

B cells are involved in the pathology of Grave's disease and autoimmune thyrioditis (Hasselbalch, Immun. Lett. 2003; 88(1): 85-86; Nielsen et al., Eur. J. Immunol., 2004; 34(1): 263-272).

### Systemic Sclerosis

There is a disturbed B cell homeostasis and increased memory B cell hyperactivity in scleroderma indicating that B cells may be a target in the treatment of scleroderma (Sato et al., Arthritis Rheum. 2004; 50(6): 1918-1927; Asano et al., Am. J. Pathol. 2004; 165(2): 641-650).

### Chronic Immune Thrombocytopenic Purpura

Inactivation of B cells is useful in the treatment of chronic immune thrombocytopenic purpura (Stasi et al., Blood 2001; 98(4): 952-957; Cooper et al., Br. J. Haematol. 2004; 125(2): 232-239; Ahmad et al., Am. J. Hematol. 2004; 77(2): 171-176).

### Other Autoimmune Disorders

B cell treatment and/or inactivation is also effective in Sjogren's syndrome autoimmune polyneuropathy (Levine and Pestronk, Neurology 1999; 52(8): 1701-1704), Wegener's granulomatosis (Specks et al., Arthritis Rheum. 2001; 44(12): 2836-2840), cold agglutinin disease associated with indolent lymphoma (Cohen et al., Leuk. Lymphoma 2001; 42(6): 1405-1408; Berensen et al., Blood 2004; 103(8): 2925-2928), idiopathic membranous neuropathy (Ruggenenti et al., J. Am. Soc. Nephrol. 2003; 14(7): 1851-1857), type II mixed cryoglobulinaemia (Zaja et al. Blood 2003; 101(10): 3827-3834), acquired factor VIII inhibitors (Wiestner et al. Blood 2002; 100(9): 3426-3428; Stasi et al., Blood 2004; 103(12): 4424-4428), fludarabine-associated immune thrombocytopenic purpura (Hegde et al., Blood 2002; 100(6): 2260-2262), refractory dermatomyositis (Levine, Arthritis Rheum. 2002; 46 Suppl. 9): 5488, pemphigus vulgaris (Dupuy et al., Arch. Dermatol. 2004; 140(1): 91-96) and myasthenia gravis (Zaja et al., Neurology 2000; 55(7): 1062-1063; Wylam et al., J Pediatr. 2003; 143 (5): 674-677; Gajra et al., Am. J. Hematol 2004; 77(2): 196-197).

Non-autoimmune conditions which may be treated in accordance with the method described herein include:

### Chronic Transplant Rejection

Since a major component of chronic rejection is antibody mediated, agents, which inhibit B cell responses may reduce the production of antibodies (Pescovitz M. D. 2004, supra). Mycophenolate mofetil and sirolimus inhibit B cell proliferation and reduce antibody formation to a neoantigen in transplant recipients (Kimball et al., Transplantation 1995; 60(12): 1379-1383; Pescovitz et al., Am. J. Transplant. 2003; 3(4): 497-500).

### Cell Lymphomas

The anti-CD20 monoclonal antibody rituximab is standard therapy in the treatment of non-Hodgkin's lymphoma and has been used in a number of other B cell malignancies, including indolent and follicular lymphoma, mantle cell lymphoma, chronic lymphocytic leukaemia, small lymphocytic lymphoma, multiple myeloma, primary cutaneous B cell lymphomas, acute lymphocytic leukaemia, Burkitt's lymphoma, HIV-associated lymphoma, primary CNS lymphoma, post-transplant lymphoproliferative disorder and Hodgkin's disease (Boye et al., Ann. Oncol. 2003; 14(4): 520-535; Avivi et al., Br. J. Cancer. 2003; 89(8): 1389-1394; Rastetter et al., Annu. Rev. Med. 2004; 55: 477-503.). This indicates a role for tranilast in the treatment of lymphomas alone and in combination with standard chemotherapy.

### Graft-Versus Host Disease (GVHD)

GVHD is characterized by a pathogenic role of B cells in this disease (Ratanatharathorn et al., Biol. Blood Marrow Transplant 2003; 9(8): 505-511).

### Acute Transplant Rejection

Infiltrating B cells play a pivotal role in acute transplant rejection (Sarwal et al., 2003, supra; Krukemeyer et al., Transplantation 2004; 78(1): 65-70). B cell MHC class II-mediated antigen presentation contributes to the pathogenesis of acute allograft rejection (Akashi et al., 1997, supra).

Other embodiments described herein are directed to methods for the treatment and/or prophylaxis of a condition characterized by aberrant or unwanted B cell activity in a subject. The method can include administering to said mammal an effective amount of one or more agents that inhibit at least one of C3aR and/or C5aR signaling in B cells. Aberrant or unwanted B cell activity should be understood as a reference to B cell responses, activity, and/or functioning, which is either not normal or which is physiologically normal but is inappropriate in that it is unwanted. Examples of such conditions include, but are not limited to, autoimmune conditions such as rheumatoid arthritis, multiple sclerosis, Crohn's disease, inflammatory bowel disease, type I diabetes, psoriasis, Graves' disease, autoimmune thyroiditis, systemic sclerosis, chronic immune thrombocytopenic purpura, autoimmune haemolytic anaemia, autoimmune polyneuropathy, Wegener's granulomatosis, cold agglutinin disease associated with indolent lymphoma, idiopathic membranous neuropathy, type II mixed cryoglobulinaemia, acquired factor VIII inhibitors, fludarabine-associated immune thrombocytopenic purpura, refractory dermatomyositis, pemphigus vulgaris and myasthenia gravis, and the non-autoimmune conditions of graft versus host disease, acute and chronic transplant rejection, septic shock, insulin resistance, apoptotic conditions, or neoplastic conditions such as multiple myeloma, B-chronic lymphocytic leukemia and other B cell neoplasias. It should be understood that the subject functioning may correspond to either or both of unwanted immunoglobulin secretion or unwanted antigen presentation. In the context of the latter, therefore, the condition may be characterized by an unwanted T cell response, the efficacy of which T cell response is linked to B cell antigen presentation. Accordingly, by down regulating the level of B cell antigen presentation, the efficacy of the unwanted T cell response may be down-regulated.

The foregoing treatment methods and uses will generally involve the administration of the pharmaceutically effective composition comprising the agent that inhibits at least one of C3aR and/or C5aR signaling in B cell to B cell site or sites. Such administration routes can include direct administration or systemic administration, by, for example, intravenous administration. “Administration”, as used herein, means provision or delivery of the agents that inhibit at least one of C3aR and/or C5aR signaling in an amount(s) and for a period of time(s) effective to attenuate B cell response.

Therapeutically effective doses of the agents that inhibit at least one of C3aR and/or C5aR signaling are readily determinable using data from an animal model. In using the agents that inhibit at least one of C3aR and/or C5aR signaling in immunotherapies, one can also draw on other published data in order to assist in the formulation of doses for clinical treatment. Any dose, or combined medicament of the agents that inhibit at least one of C3aR and/or C5aR signaling to inhibit B cell response or activity will define a useful invention.

Formulation of pharmaceutical compounds for use in the modes of administration noted above (and others) are described, for example, in Remington's Pharmaceutical Sciences (18^{th} edition), ed. A. Gennaro, 1990, Mack Publishing Company, Easton, Pa. (also see, e.g., M. J. Rathbone, ed., Oral Mucosal Drug Delivery, Drugs and the Pharmaceutical Sciences Series, Marcel Dekker, Inc., N.Y., U.S.A., 1996; M. J. Rathbone et al., eds., Modified-Release Drug Delivery Technology, Drugs and the Pharmaceutical Sciences Series, Marcel Dekker, Inc., N.Y., U.S.A., 2003; Ghosh et al., eds., Drug Delivery to the Oral Cavity, Drugs and the Pharmaceutical Sciences Series, Marcel Dekker, Inc., N.Y. U.S.A., 1999. Some variations in concentration will necessarily occur, depending on the particular complement antagonist employed, the condition of the subject to be treated and the like, and the person responsible for treatment will determine the most suitable concentration for the individual subject.

The agents that inhibit at least one of C3aR and/or C5aR signaling can be formulated for parenteral administration, systemic administration, and/or local or topical administration. For example, agents that inhibit C3aR and/or C5aR signaling can be administered to B cells in the blood or vasculature by intravenous injection or using, for example, a medical device that can be delivered to the vasculature.

Pharmaceutical compositions of the agent will generally include an amount of the agent admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, incorporated herein by reference. Moreover, for human administration, preparations should meet sterility, pyrogenicity, general safety and purity standards as required by FDA Office of Biological Standards.

The pharmaceutical composition can be in a unit dosage injectable form (e.g., solution, suspension, and/or emulsion). Examples of pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (e.g., glycerol, propylene glycol, liquid polyethylene glycol, and the like), suitable mixtures thereof and vegetable oils.

Proper fluidity can be maintained, for example, by the use of a coating, such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various amounts of the other ingredients, as desired.

Pharmaceutical “slow release” capsules or “sustained release” compositions or preparations may be used and are generally applicable. Slow release formulations are generally designed to give a constant drug level over an extended period and may be used to deliver the agent.

The medical device for delivery of the agent can include, for example, endovascular medical devices, such as intracoronary medical devices. Examples of intracoronary medical devices can include stents, drug delivery catheters, grafts, and drug delivery balloons utilized in the vasculature of a subject.

In other embodiments described herein, a B cell response (e.g., growth, proliferation, and/or antibody production) can be promoted or stimulated by administering to the B cell an agent that promotes or stimulates C3aR and/or C5aR signaling of the B cells. The agent can be selected from the group consisting of C3, C5, C3a, C5a, a C3aR agonist, a C5aR agonist, a DAF antagonist, or combination thereof.

Promotion or stimulation of C3aR and/or C5aR activation in B cells can be used to stimulate, promote, and/or enhance growth, viability, proliferation, and antibody production of the B cells.

In some embodiments, an agent that promotes or stimulates C3aR and/or C5aR signaling can be used for inducing an immunological response and/or antibody production in a mammal (e.g., human). The agent can be provided with a vaccine that can be inoculated in an individual to produce antibody and/or a B cell immune response. The response can be adequate to protect said individual from infection, particularly bacterial or viral infection. Thus, the immunological response may be used therapeutically or prophylatically.

The vaccine antigen can include but is not limited to bacterial, viral, parasitic, allergens, autoantigens and tumor associated antigens. Particularly, the antigen can include protein antigens, peptides, whole inactivated organisms, and the like.

Specific examples of antigens that can be used in the invention include antigens from hepatitis A, B, C or D, influenza virus, Listeria, Clostridium botulinum, tuberculosis, tularemia, Variola major (smallpox), viral hemorrhagic fevers, Yersinia pestis (plague), HIV, herpes, pappilloma virus, and other antigens associated with infectious agents. Other antigens include antigens associated with a tumor cell, antigens associated with autoimmune conditions, allergy and asthma. Administration of such an antigen in conjunction with the subject immune combination can be used in a therapeutic or prophylactic vaccine for conferring immunity against such disease conditions.

In some embodiments, the agent that promotes or stimulates C3aR and/or C5aR signaling can be used to treat an individual at risk of having an infection or has an infection by including an antigen from the infectious agent. An infection refers to a disease or condition attributable to the presence in the host of a foreign organism or an agent, which reproduce within the host. A subject at risk of having an infection is a subject that is predisposed to develop an infection. Such an individual can include for example a subject with a known or suspected exposure to an infectious organism or agent. A subject at risk of having an infection can also include a subject with a condition associated with impaired ability to mount an immune response to an infectious agent or organism, for example a subject with a congenital or acquired immunodeficiency, a subject undergoing radiation or chemotherapy, a subject with a burn injury, a subject with a traumatic injury, a subject undergoing surgery, or other invasive medical or dental procedure, or similarly immunocompromised individual.

Infections which may be treated or prevented with the agent that promotes or stimulates C3aR and/or C5aR signaling include bacterial, viral, fungal, and parasitic. Other less common types of infection also include are rickettsiae, mycoplasms, and agents causing scrapie, bovine spongiform encephalopathy (BSE), and prion diseases (e.g., kuru and Creutzfeldt-Jacob disease). Examples of bacteria, viruses, fungi, and parasites that infect humans are well known. An infection may be acute, subacute, chronic or latent and it may be localized or systemic. Furthermore, the infection can be predominantly intracellular or extracellular during at least one phase of the infectious organism's agent's life cycle in the host.

Bacteria infections against which the subject agent, vaccines and methods may be used include both Gram negative and Gram positive bacteria. Examples of Gram positive bacteria include but are not limited to Pasteurella species, Staphylococci species, and Streptococci species. Examples of Gram negative bacteria include but are not limited to Escherichia coli, Pseudomonas species, and Salmonella species. Specific examples of infectious bacteria include but are not limited to Heliobacter pyloris, Borrelia burgdorferi, Legionella pneumophilia, Mycobacteria spp. (for example M. tuberculosis, M. avium, M. intracellilare, M. kansaii, M. gordonae), Staphylococcus aureus, Neisseria gonorrhoeae, Neisseria meningitidis, Listeria monocytogeners, Streptococcus pyogenes, (group A Streptococcus), Streptococcus agalactiae (Group B Streptococcus), Streptococcus (viridans group), Streptococcus faecalis, streptococcus bovis, Streptococcus (anaerobic spp.), Streptococcus pneumoniae, pathogenic Campylobacter spp., Enterococcus spp., Haemophilus influenzae, Bacillus anthracis, Corynebacterium diptheriae, Corynebacterium spp., Erysipelothrix rhusiopathie, Clostridium perfringens, Clostridium tetani, Enterobacter aerogenes, Klebsiella pneumoniae, Pasteurella multocida, Bacteroides spp., Fusobacterium nucleatum, Streptobacillus moniliformis, Treponema pallidum, Treponema pertenue, Leptospira, Rickettsia, and Actinomyces israelii.

Examples of viruses that cause infections in humans include but are not limited to Retroviridae (for example human deficiency viruses, such as HIV-1 (also referred to as HTLV-III), HIV-II, LAC or IDLV-III (LAV or HIV-III and other isolates such as HIV-LP, Picornaviridae (for example poliovirus, hepatitis A, enteroviruses, human Coxsackie viruses, rhinoviruses, echoviruses), Calciviridae (for example strains that cause gastroenteritis), Togaviridae (for example equine encephalitis viruses, rubella viruses), Flaviviridae (for example dengue viruses, encephalitis viruses, yellow fever viruses) Coronaviridae (for example coronaviruses), Rhabdoviridae (for example vesicular stomata viruses, rabies viruses), Filoviridae (for example Ebola viruses) Paramyxoviridae (for example parainfluenza viruses, mumps viruses, measles virus, respiratory syncytial virus), Orthomyxoviridae (for example influenza viruses), Bungaviridae (for example Hataan viruses, bunga viruses, phleoboviruses, and Nairo viruses), Arena viridae (hemorrhagic fever viruses), Reoviridae (for example reoviruses, orbiviruses, rotaviruses), Bimaviridae, Hepadnaviridae (hepatitis B virus), Parvoviridae (parvoviruses), Papovaviridae (papilloma viruses, polyoma viruses), Adenoviridae (adenoviruses), Herpeviridae (for example herpes simplex virus (HSV) I and II, varicella zoster virus, pox viruses) and Iridoviridae (for example African swine fever virus) and unclassified viruses (for example the etiologic agents of Spongiform encephalopathies, the agent of delta hepatitis, the agents of non-A, non-B hepatitis (class 1 enterally transmitted; class 2 parenterally transmitted such as Hepatitis C); Norwalk and related viruses and astroviruses).

Examples of fungi include Aspergillus spp., Coccidoides immitis, Cryptococcus neoformans, Candida albicans and other Candida spp., Blastomyces dermatidis, Histoplasma capsulatum, Chlamydia trachomatis, Nocardia spp., and Pneumocytis carinii.

Parasites include but are not limited to blood-borne and/or tissue parasites such as Babesia microti, Babesi divergans, Entomoeba histolytica, Giarda lamblia, Leishmania tropica, Leishmania spp., Leishmania braziliensis, Leishmania donovdni, Plasmodium falciparum, Plasmodium malariae, Plasmodium ovale, Plasmodium vivax, Toxoplasma gondii, Trypanosoma gambiense and Trypanosoma rhodesiense (African sleeping sickness), Trypanosoma cruzi (Chagus' disease) and Toxoplasma gondii, flat worms, and round worms.

The agent that promotes or stimulates C3aR and/or C5aR signaling of the B cells can be administered systemically by, for example, intravenous injection to the subject.

Pharmaceutical compositions will generally include an amount of the agent that promotes or stimulates C3aR and/or C5aR signaling of the cells or variants thereof admixed with an acceptable pharmaceutical diluent or excipient, such as a sterile aqueous solution, to give a range of final concentrations, depending on the intended use. The pharmaceutical composition can be in a unit dosage injectable form (e.g., solution, suspension, and/or emulsion).

The following examples are for the purpose of illustration only and are not intended to limit the scope of the claims, which are appended hereto.

### EXAMPLE

Deficient or deregulated B cell responses are connected with hypo-immune and auto-immune disorders and B cell responses that are elicited by vaccines are needed to prevent numerous infectious and other diseases. Among disorders connected with inadequate B cell responses are Bruton's-hypoglobulinemia and HIV-AIDs, and among those connected with abnormally heightened B cell responses are systemic lupus erythematosus (SLE) and myasthenia gravis (MG). Protective vaccination strategies currently are intensively being sought for Ebola virus and for Malaria falciparum among many other infectious agents. Consequently, the ability to repress or to boost B cell responses has broad clinical relevance.

Our experiments have uncovered a heretofore unrecognized process that controls B cell responses. These studies have shown that the complement anaphylatoxins C3a and C5a, generally believed to derive from serum complement proteins and function principally in innate immunity, are in fact endogenously synthesized by B cells themselves and feedback through C3a and C5a receptors (C3aR/C5aR) on their surfaces to transduce signals needed for B cell activation. This autocrine C3aR/C5aR signaling is required for the survival and growth signaling by the B cell growth factors BAFF and APRIL. It also is required for the proliferation of B cells in response to antigen and CD4⁺ T cell help as well as for B cell production of inflammatory cytokines including IL-6. Moreover, it is required for class switch recombination (CSR) which gives rise antibodies of different isotypes, i.e., IgG1, 2, 3, 4, IgE, and IgA, that participate in multiple immune processes.

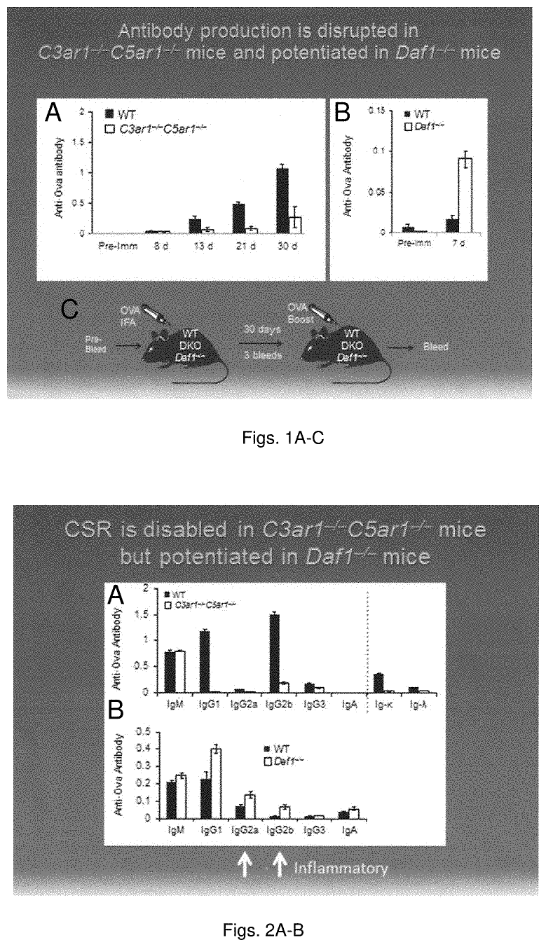

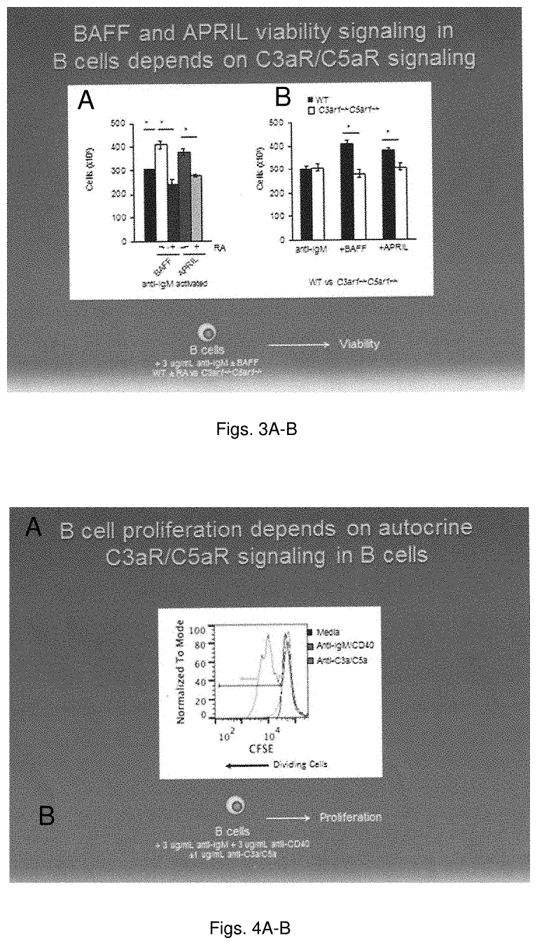

In studies of antibody production, we found that immunization of mice doubly deficient in C3aR and C5aR (C3ar1^{-/-}C5ar1^{-/-} mice) with ovalbumin (ova) elicited IgM anti-ova Abs but not other anti-ova isotypes. (FIGS. 1(A-C) and FIGS. 2(A-B)). Disabling B cell C3aR/C5aR signaling suppressed BAFF and APRIL B cell growth, whereas added C5a had the opposite effect. (FIGS. 3(A-C)). B cell proliferation, IL-6 production and CD40 expression that leads to BcI-6, and AID up-regulation, as well as CD4⁺ cell IL-21 production were similarly inhibited. (FIGS. 4(A-B)). Following ova immunization, recipients of adoptively transferred C3ar1^{-/-}C5ar1^{-/-} B cells produced reduced IgM Ab and no other isotypes. Notably, purified B cells cultured under activating conditions exhibited surface C3 antigen and CD19 phosphorylation, a signaling step involved in both the primary and secondary B cell response.

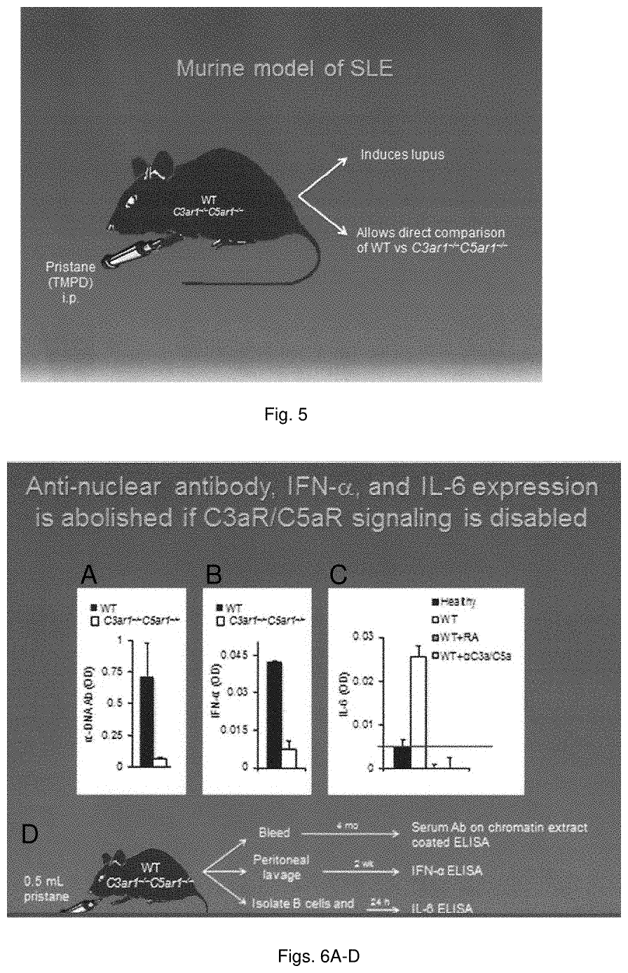

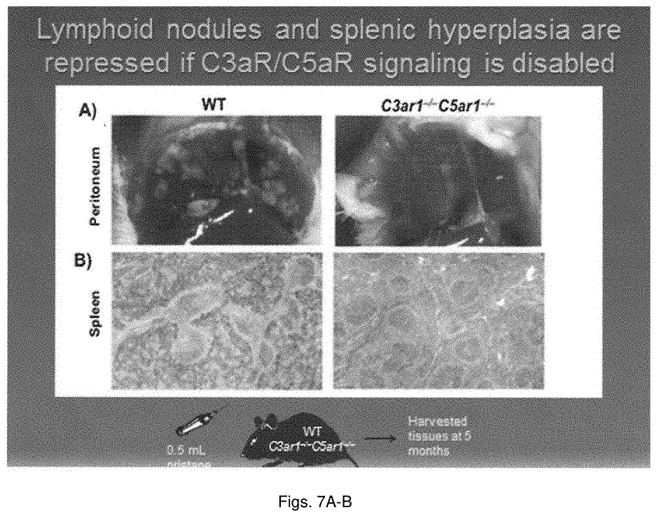

In in vivo studies in the pristane induced model of SLE, we found that disease was virtually abolished in C3ar1^{-/-}C5ar1^{-/-} mice. (FIGS. 5, 6, and 7). Heightened IL-6 production, IFN-a production, and anti-nuclear antibody production, three hallmarks of SLE, were suppressed. Taken together, the data show that autocrine C3aR1C5aR signaling in B cells is integral to both primary and secondary Ab responses and that inhibition of this signaling ameliorates SLE. Other experiments conversely showed that potentiated C3aR/C5aR signaling in B cells augments the antibody response, a finding centrally relevant to improved vaccine development.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A method of treating a B cell mediated disorder in a subject in need thereof, the method comprising:
administering to B cells of the subject at least one agent that inhibits C3aR and/or C5aR signaling of the B cells.

2. The method of claim 1 wherein the at least one agent comprises a C3a antagonist or C3aR antagonist and the C5a antagonist or C5aR antagonist.

3. The method of claim 2, the C3a antagonist or C3aR antagonist and the C5a antagonist or C5aR antagonist being selected from the group consisting of a small molecule, a polypeptide, and a polynucleotide.

4. The method of claim 3, the polypeptide comprising an antibody directed against at least one of C3a, C5a, C3aR, or C5aR.

5. The method of claim 3, the polynucleotide comprising a small interfering RNA directed against a polynucleotide encoding at least one of C3 , C5 , C3aR, or C5aR.

6. The method of claim 2, the step of administering the complement antagonist includes administering to the B cells a C3aR antagonist and a C5aR antagonist.

7. The method of claim 6, wherein the C3aR antagonist and the C5aR antagonist are antibodies.

8. The method of claim 1, the step of administering the at least one complement antagonist includes administering to the B cells a C3a antagonist and a C5a antagonist.

9. The method of claim 7, wherein the C3a antagonist and C5a antagonist are antibodies.

10. The method of claim 1, wherein the B cell mediated disorder is an autoimmune disease selected from the group consisting of systemic lupus erythematosus, Sjogren's syndrome, scleroderma, rheumatoid arthritis, juvenile idiopathic arthritis, graft versus host disease, dermatomyositis, type I diabetes mellitus, Hashimoto's thyroiditis, Graves's disease, Addison's disease, celiac disease, Crohn's Disease, pernicious anaemia, Pemphigus vulgaris, Vitiligo, autoimmune haemolytic anaemia, idiopathic thrombocytopenic purpura, giant cell arteritis, Myasthenia gravis, multiple sclerosis (MS), preferably relapsing-remitting MS (RRMS), glomerulonephritis, Goodpasture's syndrome, bullous pemphigoid, colitis ulcerosa, Guillain-Barre syndrome, chronic inflammatory demyelinating polyneuropathy, Anti-phospholipid syndrome, narcolepsy, sarcoidosis, and Wegener's granulomatosis.

11. A method of treating systemic lupus erythematosus in a subject in need thereof, the method comprising:
administering to B cells of the subject at least one agent that inhibits C3aR and/or C5aR signaling of the B cells.

12. The method of claim 11 wherein the at least one agent comprises a C3a antagonist or C3aR antagonist and the C5a antagonist or C5aR antagonist.

13. The method of claim 12, the C3a antagonist or C3aR antagonist and the C5a antagonist or C5aR antagonist being selected from the group consisting of a small molecule, a polypeptide, and a polynucleotide.

14. The method of claim 13, the polypeptide comprising an antibody directed against at least one of C3a, C5a, C3aR, or C5aR.

15. The method of claim 13, the polynucleotide comprising a small interfering RNA directed against a polynucleotide encoding at least one of C3, C5, C3aR, or C5aR.

16. The method of claim 12, the step of administering the complement antagonist includes administering to the B cells a C3aR antagonist and a C5aR antagonist.

17. The method of claim 16, wherein the C3aR antagonist and the C5aR antagonist are antibodies.

18. The method of claim 11, the step of administering the at least one complement antagonist includes administering to the B cells a C3a antagonist and a C5a antagonist.

19. The method of claim 17, wherein the C3a antagonist and C5a antagonist are antibodies.
